(19) United States
(12) Patent Application Publication
(10) Pub. No.: US 2015/0119321 A1
(43) Pub. Date: Apr. 30, 2015

(54) System and Method of Losing Weight

(71) Applicant: Linda Duerler FINCH, (US)

(72) Inventor: Linda Duerler Finch, Winston-Salem, NC (US)

(21) Appl. No.: 14/390,274

(22) PCT Filed: Apr. 5, 2013
(86) PCT No.: PCT/US13/00104

§ 371 Date: Oct. 2, 2014

Related U.S. Application Data
(60) Provisional application No. 61/620,637, filed on Apr. 5, 2012.

(51) Int. Cl. G09B 19/00; A61K 31/675; A61K 31/525; A61K 31/194; A61K 38/24; A61K 31/714

(52) U.S. Cl. CPC G09B 19/0092; A61K 38/24; A61K 31/714; A61K 31/525; A61K 31/194; A61K 31/675
USPC 514/5.3; 434/127; 514/52; 514/574; 514/89

(57) ABSTRACT

A unique combination of weight loss modalities that produce 20-40 pounds of weigh loss in a six week treatment period. Treatment includes a meal plan chosen to provide adequate protein and nutrition during dieting and to assist metabolism of toxins created from the release of stored fat, high amounts of fiber to assist in collecting toxins and fat in the elimination system and to encourage peristaltic action in the bowel, a bowel toner to further increase peristaltic action in the bowel, stimulating liver functions of fat and toxins. The weight loss portion of the treatment is limited to six week segments, and added to the plurality of modalities comprising the invention, to further support the body during weight loss. By supporting the body during weight loss, his invention(s) avoids the “plateau” routinely experienced by existing weight loss systems, when the metabolism slows to avoid further damage from ultra-low calories, medications, bariatric surgeries, and other stressful programs. A stabilization period is used to maintain the weight lost, and dramatically reduce the incidence of rebound weight gain.

### RELATED APPLICATIONS

The present application is a continuation-in-part application of United States provisional patent application, U.S. Pat. No. 6,162,637, filed Apr. 5, 2012, for SYSTEM AND METHOD OF LOSING WEIGHT, by LINDA DUERLER FINCH, included by reference herein and for which benefit of the priority date is hereby claimed.

### FIELD OF THE INVENTION

The present invention relates to a physician prescribed weight loss treatment, and, more particularly, to a treatment that is a unique combination of weight loss modalities that provide faster weight loss, more reduction in inches/pound lost, weight loss without hunger or fatigue, and substantially reduced rebound weight gain.

### BACKGROUND OF THE INVENTION

Despite the availability of a plurality of methods of weight control, including low calorie diets, exercise, medications, surgeries and more, obesity and related health problems continue to increase. More than one-third of U.S. adults (35.7%) are obese, and an additional one-third is overweight. The CDC states that obesity increases the risk of developing coronary heart disease, type 2 diabetes, cancers (endometrial, breast, and colon), hypertension (high blood pressure), dyslipidemia, stroke, liver and gallbladder disease, sleep apnea and respiratory problems, osteoarthritis, and gynecological problems, including abnormal menses, infertility).

Obesity-related conditions include some of the leading causes of preventable death. A team of scientists supported in part by a component of NIH, projects that over the next few decades, life expectancy for the average American could decline by as much as 5 years, unless aggressive efforts are made to slow rising rates of obesity. One study, cited by the team, indicates obesity in U.S. adults has increased about 50 percent per decade since 1980, people who are severely obese—with a BMI greater than 45—live up to 20 years less than people who are not overweight. Some researchers have estimated that obesity causes about 300,000 deaths in the U.S. annually. In 2008, medical costs associated with obesity were estimated at $147 billion; the medical costs for people who are obese were $1,429 higher than those of normal weight.

There exists an ongoing need to stop, if not reverse, the rate of growth of the condition of obesity. Weight loss diets, programs, and treatments have failed to stem this epidemic currently overtaking the U.S. and spreading across the world. Weight loss methods currently available are shown to be ineffective at weight reduction, damaging to the dieter physically and emotionally, have significant side effects, and effect very poor compliance to the programs.

Current weight loss choices do not provide an effective solution to the epidemic of obesity and related disease. Weight loss treatments abound, and include 1. Calorie and nutrient restricted diet programs such as Weight Watchers, Nutrisystem, Atkins, and others; 2. exercise; 3. amphetamines, phentermine and other prescription medications, 4. various types of bariatric surgery, 5. the HCG diet, 6. and a permanent and complete change to a healthier lifestyle, meals, and exercise.

1. Calorie/Nutrient Restricted Programs abound, all of which require the dieter to eat less and exercise more, or to limit particular nutrients such as fat or carbohydrates. These starvation or semi-starvation diets, such as Weight Watchers Nutrisystem, Atkins, and others require semi-starvation meal plans, of 1,000-1,600 calories. A descriptive study of weight loss maintenance of many diets, (Sarlio-Rissanen, Kaprio, Int J Obes Relat Metab Disord. 2000 January;24(1):116-250), found that only 6% of all overweight individuals lost and maintained at least a 5% weight loss, or reversely stated, 94% of all attempts to lose weight fail. Dr. Robert H Eckel, in JAMA. 2005; 293(1):96-97), summarized the randomized trial of diets by Dansinger et al 3 in the same issue. The trial, trying to determine which diet works best, found the primary outcome of weight loss at 1 year was modest—2-4 Kg (4.4-6.6 pounds), and there was no difference between the low-carbohydrate approach of Atkins, the high-protein low-glycemic load approach of Sears (the Zone diet), the very low-fat approach of Ornish, and the low-calorie/portion-size approach of Weight Watchers. The results from all of them are wanting. Each of these diets deprives dieters of essential nutrients, causing damage to the metabolism and future weight gain.

In addition, studies demonstrate physical and psychological damage from these diets, including a slowing of the metabolism, causing more weight gain. The Minnesota Starvation Experiment studied effects of prolonged, famine-like semi-starvation diets on healthy men, during a 6-month semi-starvation period, where dietary intake was cut to approximately 1,560 calories per day; exceeding the calories allowed by many popular diet programs, such as Weight Watchers and Nutrisystem, of 1,000 to 1,600. The study confirmed that prolonged semi-starvation produces significant increases in depression, hysteria and hypochondriasis. Participants exhibited a preoccupation with food, both during the starvation period and after. Some experienced bouts of binge-eating, the inability to satisfy hunger, and experienced episodes of uncontrolled eating, after returning to normal diets. Participants showed signs of social withdrawal and isolation; a decline in concentration, comprehension, and judgment; and marked declines in physiological processes indicative of a decrease in each subject's basal metabolic rate.

Dr. Diane Schwarzbein and Dr. Mark Hymen's research reveals that the limbic system is designed, to protect the body from starvation, a reaction learned from thousands of generations, which lived without consistent food supplies. They believe once the body perceives it is dieting, an “insulin fat storage system” is activated. In the first 10-14 days, glycogen stores are used and some fat is lost. Then the body starts to use bone and muscle for fuel for the brain and other functional metabolic processes. The metabolism slows to protect the bone and muscle, and weight loss slows. If the deficient diet continues, damage increases. When a goal weight is achieved, the body attempts to recover by regaining the weight lost, and more.

The more radical the diet, with liquids, shakes, or 500 calorie HCG programs, the faster the damage. Repeated or yo-yo dieting exacerbates the problem, leaving people with a much higher weight, damaged metabolism, and degraded body composition.

2. Exercise is frequently recommended, and while exercise has many health benefits, studies show that dieters are frequently not compliant, and weigh loss is minimal, if any.

3. Prescription medications have done nothing to stop the epidemic of obesity. Phentermine, amphetamines and prescription drugs have been prescribed by physicians to effect “temporary” weight loss. Fen-phen, fenfluramine/phentermine, was marketed heavily until potentially fatal side effects became evident. Phentermine is frequently used for weight loss today, but is only approved for short-term treatment of obesity, as an appetite suppressant, should be combined with exercise, diet, and behavioral modification. Side effects include tachycardia, elevated blood pressure, and heart rate, palpitations, restlessness, and insomnia. Phentermine has the potential to cause psychological dependence. After short-term use, tolerance begins and can be, and usually is, followed by rebound weight gain.

Xenical, blocks fat from being absorbed but may give a dieter stomach pain uncontrollable diarrhea, block in urine, paid in lower back, no urinating, confusion, swelling, weight gain, fast heart rate, and others: Qsymia is a combination of phentermine and topiramate and includes side effects of pounding heartbeat, depression, confusion, blurred vision, headache, hunger, low blood sugar, severe paid in the lower back, blood in urine, unable to urinate, uneven heart rate, and others. Belviq suppresses appetite and may be habit forming, and has other side effects that include shortness of breast, slow heart rate, body aches, flu symptoms, breast swelling and nipple discharge in men and women, agitation, hallucinations, very rigid muscles, uneven heart beat and others. These and other medications all require a patient to forever diet and exercise.

4. Gastric bypass and other weight loss surgeries are performed to limit the amount of food a person can ingest. The risk of death at the time of surgery is twice that of the average for other surgeries. Side effects include body aches, feeling as if one has the flu, hair loss, additional surgeries, bowel obstruction, dumping syndrome, gallstones, vomiting, low blood sugar, malnutrition, stomach perforation, ulcers, and death. Patients rarely achieve a normal BMI, but the pounds lost do improve some medical conditions of patients. Rebound weigh gain is common from these surgeries.

5. HCG diet. The HCG diets only achieve weight loss an ultra-low calories diet, 500 to 700 calories per day, is consumed during the treatment. Both prescription HCG, and homeopathic types of “over-the-counter” HCG (non prescription) require the ultra-low calorie diet. Patients lose weight quickly, but ultimately this treatment has the same results as described in the low calorie “starvation or semi-starvation” diets. If a dieter eats more than 500-700 calories per day with an HCG diet, they do not achieve weight loss.

6. For persons with the extreme willpower to eat well, include a good balance of nutrients, not eat the very low calorie diets, exercise moderately, and live a healthy life style, this solution is somewhat successful. The dieter must maintain this situation to maintain the lower weight for a year or more. The slow achievement of health improvements and weight loss result in few persons that are able to use this method with some success. This option is not one most dieters can maintain.

Dr. Eckel tells us, ‘One important similarity between the Dansinger et al 3 trial and the other studies is a high dropout rate. In the study 42% of the study participants were no longer adhering to their diets by 12 months, and those participants who did complete the trial had a weight reduction of only 5 kg or 5% from baseline weight. Adherence rates were poor for all 4 diets.”

The review of the study by Dr. Eckel further notes, “a concern historically related to the dietary treatment of obesity is estimated caloric intake at baseline. The reported average caloric intake in the Dansinger et al 3 trial was only 2059 kcal for participants whose mean weight was 100 kg (220 lb). This is well below a very conservative estimate of the 2860 calories needed. Losing weight and sustaining weight reduction are very difficult. Beyond a lack of willpower is a plethora of biological explanations that challenge the dieter after and during weight loss.” These problems cause the plateaus and rebound weight gain prevalent in existing weight loss choices.

The failure of existing technology to produce quick and effective weight loss is evident in continued rise in the rate of obesity, despite the use and availability of these methods. More and more people are becoming overweight, creating and worsening health issues, all exacerbated by obesity.

In view of the above circumstances, it would be advantageous to provide a weigh loss program that produces significant weight loss in a short six week treatment period, in which a patient was not hungry or tired, improving compliance and achieving more weight loss during treatment; and

It would also be advantageous to provide a weight loss program that provides adequate protein and nutrition, adjusted for each individual, to assist metabolizing toxins from the release of stored fat, reducing the stress of dieting upon the body, and not triggering a slowing of the metabolism, and still able to reduce weight at an average many times faster than previously existing programs.

It would further be advantageous to provide a weight loss program that created a significant loss of inches and size without the use of amphetamines or other medication, creating a program where there are no significant negative side effects.

It would also be advantageous to provide a weight loss program that provides a method for stabilizing the dieter at the newly achieved lower weight, and reducing the typical rebound weight gain experienced from current programs.

### SUMMARY OF THE INVENTION

In accordance with the present invention, there is provided a unique combination of weight loss modalities that may produce 20-40 pounds of weigh loss in a six-week period, without the hunger or the fatigue usually experienced during weight loss. This new combination of common weight loss modalities supports the body during a limited treatment period of only six weeks, and provides consistent weight loss without the usual plateau at 10-14 days. A nutritionally adequate meal plan of high protein, low fat, and moderate carbohydrates, matched to the metabolic function of each patient, is available to reduce stress and toxicity of dieting, and is still able to consistently achieve weight loss of 20-40 pounds in just six weeks, at a rate of loss two to three times higher than existing treatments. The meal plan is designed and modified for each patient based upon weight, age, height, sex, and physical activity.

The body will “plateau” during weight loss. The metabolism will slow, and further weight loss is stopped, regardless of diet or exercise. This is demonstrated with the results of the typical weight loss from a bariatric surgery. A patient will lose some weight, not enough to achieve a normal BMI, and as some point weight loss will stop, the patient will regain part of their weight, and even though caloric intake remains at the same level as during weight loss, the same restriction of the ability to ingest food still exists, the patient will stop losing weight. Studies show that a loss of 10% of body weight can reduce caloric requirements by as much as 20%, as metabolism slows to prevent damage to bone and muscle due to “semi-starvation”. Diets simply stop working.

This invention(s) is plurality of components, combined in a specific manner, to allow the body to be protected with adequate nutrition, support, and cleansing, and not excite the protective mechanisms of typical stressful dieting. The activation, enhancement, and support of the body's system of elimination provide for a higher elimination of fats from the system. This physician prescribed treatment combines large amounts of soluble fiber, B12, folic acid, herbal stimulants to create significant peristaltic action, support of the liver during the treatment, a small amount of HCG, and minerals. This combination allows the body to shed weight quickly, without the usual plateau at 10-14 days, and encourages patient compliance by producing fast and easy weight loss, without hunger or fatigue. Results are improved by the physiological support provided with the combination, and the ease of treatment.

The components, while many are used separately in common weight loss programs with limited or poor results, when combined in a unique way to achieve weight loss at a rate 2-3 times more than any component used alone, without the hunger and fatigue common with use of existing programs, and when a method to stabilize the body after weight loss is added, typical rebound weight gain is significantly reduced. Exercise is not required as part of the treatment.

HCG Diets only work when the dieter consumes ultra-low calories, 500 to 750 per day. With more nutrition or protein, the use of HCG does not allow weight loss.

The use of HCG as a component of the program, combined with a nutritionally adequate meal plan and other components of the invention, provides faster weight loss, better reshaping of the body, and all without hunger or fatigue. This result is only possible because of the new and unique combination of components. The invention herein avoids this “plateau” by providing adequate nutritional, and supplement support, removal of toxins, increased peristaltic activity to remove waste and stimulate liver activity, encouraging the liver to pump more of its fat and waste into the colon.

These, and the other components, combined with a short treatment periods of six weeks of weight loss, prevent the slowing of the metabolism and a plateau that stops weight loss. During the stabilization period, when weight loss is stopped and any variation in weight results in an immediate return to the new weight achieved during the weight loss period, establishes a new weight, and allows a return to normal meals without the typical rebound weight gain.

The invention(s) reshapes the body by removing a higher percentage of fat that previous weight loss programs. The inches lost are increased. See fi

Patients reported consistently that they are not hungry or tired during any portion of the treatment.

### BRIEF DESCRIPTION OF THE DRAWINGS

A complete understanding of the present invention may be obtained by reference to the accompanying drawings, when considered in conjunction with the subsequent, detailed description, in which:

FIG. 1 is an overview view of a combined group of components, which create one embodiment of the system and method of losing weight, by combining individual modalities in a unique way to achieve unusually fast and effective weight loss.

FIG. 2 is a method to determine which meal plan in FIG. 3 should be used for each patient.

FIG. 3 is a meal plan that may be used in one embodiment of the invention.

FIG. 4 is a list of patients, redacted per HIPPA regulations, that records the pounds and inches lost for patients choosing to report information when treated.

FIG. 5 is a list of case studies of individual success.

FIG. 6 is a photo record of weekly weight loss demonstrating a substantial change in shape and loss of inches at the waist of the patient.

For purposes of clarity and brevity, like elements and components will bear the same designations and numbering throughout the Figures.

### EXEMPLARY DESCRIPTION OF THE PREFERRED EMBODIMENT

Since other modifications and changes varied to fit particular operating and functional requirements and environments will be apparent to those skilled in the art, the invention is not considered limited to the example chosen for purposes of disclosure, and covers all changes and modifications which do not constitute departures from the true spirit and scope of this invention.

For the purposes of promoting an understanding of the principles of the invention, reference will now be made to the exemplary embodiments illustrated in the drawing, and specific language will be used to describe the same. It will nevertheless be understood that no limitation of the scope of the invention(s) is thereby intended. Any alterations and further modifications of the inventive features illustrated herein and any additional applications of the principles of the invention(s) as illustrated herein, which would occur to one skilled in the relevant art and having possession of this disclosure, are to be considered within the scope of the invention(s).

Reference throughout this specification as an “embodiment,” an “example” or similar language means that a particular feature, structure, characteristic, or combinations thereof described in connection with the embodiment is included in at least one embodiment of the present invention(s). This, appearances of the phrases an “embodiment,” and “example,” and similar language throughout this specification may, but do not necessarily, all refer to the same embodiment, to different embodiments, or to one or more of the figures. Additionally, reference to the working “embodiment”, “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly, or explicitly.

As used herein, “comprising,” “including,” “containing,” “is”, “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of”.

Reference throughout this specification to features, advantages, or similar language does not imply that all of the features and advantages that may be realized with the present invention(s) should be or are in any single embodiment of the invention(s). Rather, language referring to the features and advantages is understood to mean that a specific feature, advantage, or characteristic described in connection with an embodiment is included in at least one embodiment of the present inventions(s). Thus, discussion of the features and advantages, and similar language, throughout this specification may, but do not necessarily, refer to the same embodiment.

Furthermore, the described features, advantages, and characteristics of the invention(s) may be combined in any suitable manner in one or more embodiments. One skilled in the relevant art will recognize that the invention(s) can be practiced without one or more of the specific features or advantages of a particular embodiment. In other instances, additional features and advantages ma be recognized in certain embodiments that may not be present in all embodiments of the inventions(s).

These features and advantages of the present invention(s) will become more fully apparent from the following description or may be learned by the practice of the invention(s) as set forth hereinafter.

According to one embodiment of the invention, the system and method may include a physician prescribed and physician managed weight loss program. The program may include a meal plan including an amount of protein based on weight, sex, level of activity, etc., fiber and supplements. The program may include a specific period of treatment to limit stress upon the body experienced with any calorie restriction, and a stabilization period where large amounts of specific fatty acids and complete fats are provided in specific amounts for a specific period. The end result is weight loss that is faster than conventional methods, and reduces typical rebound weight gain.

The system and method may include a combination of vitamin supplements, large amount of fiber, small amount of HCG, specific quantity of protein the diet, stabilization program. The system and method may provide quick weight loss, a comfortable and happy feeling during weight loss, not a tired feeling during weight loss, a high energy consistent weight plan, an easy to follow system and method, a stabilized insulin level, less likely to have rebound weight, more of a permanent change, a stabilization weight. The program allow for heavy exercise, not allowed with other diets using HCG.

After the system and method is over, the system and method reduces cravings for carbohydrates. The system and method may include adding more fiber, changing the HCG dosage, adding more/other supplements, adding methionine, choline. The treatment does not require exercise to enhance or create weight loss.

It is understood that the included embodiments are only illustrative of the application of the principles of the present invention(s). The present invention(s) may be embodied in other specific forms without departing from its spirit or essential characteristics. The described embodiment is to be considered in all respects only as illustrative and not restrictive. The scope of the invention(s) is, therefore, indicated by the appended claims rather than by the description. All changes which come within the meaning and range of equivalency of the claims are to be embraced within their scope.

This, while the present invention(s) has been fully described above with particularity and detail in connection with what is presently deemed to be the most practical and preferred embodiment of the invention(s), it will be apparent to those of ordinary skill in the art that numerous modifications, including, but not limited to, variations in size, quantity, materials, shape, form, function, manner of treatment or operation, assembly, combination, and use may be made, without departing from the principles and concepts of the invention(s) as set forth in the claims. Further, it is contemplated that an embodiment may e limited to consist of or to consist essentially of one or more of the features, functions, structures, methods described herein.

Since other modifications and changes varied to fit particular operating and functional requirements and environments will be apparent to those skilled in the art, the invention is not considered limited to the example chosen for purposes of disclosure, and covers all changes and modifications which do not constitute departures from the true spirit and scope of this invention.

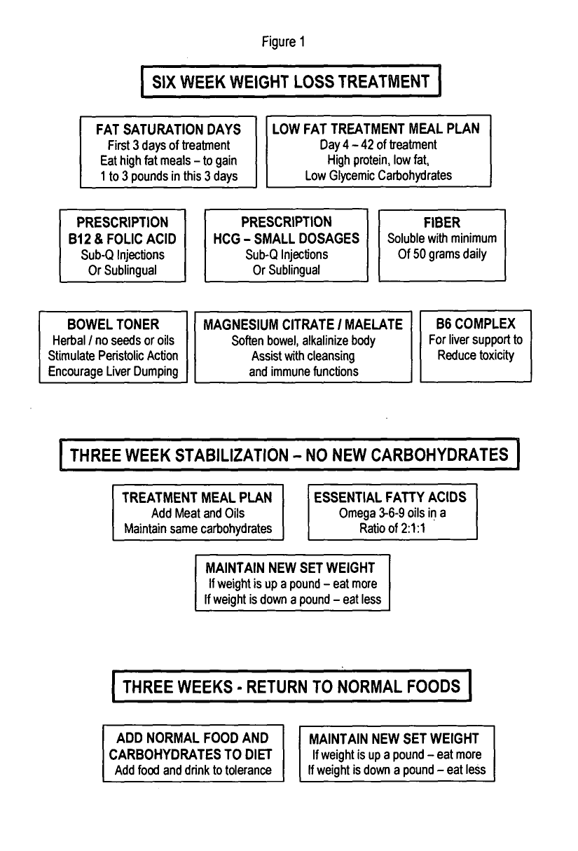

FIG. 1 is an overview of one embodiment of the invention.

Multiple methods and features of the present invention are novel in relation to current practice. Although fiber, B12, HCG, and limited calories and other methods are used separately, it is the use of the components in a unique combination that produces results that are more beneficial that other available programs.

As described in the drawing and text herein, one embodiment of the treatment consists of a “six week treatment period”, a “three week stabilization period”, and a “three week period to return to normal foods”.

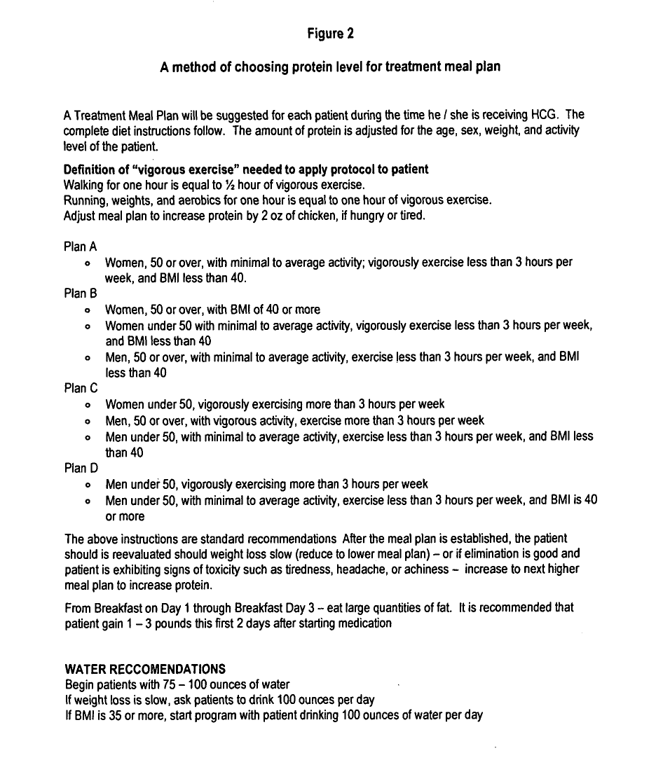

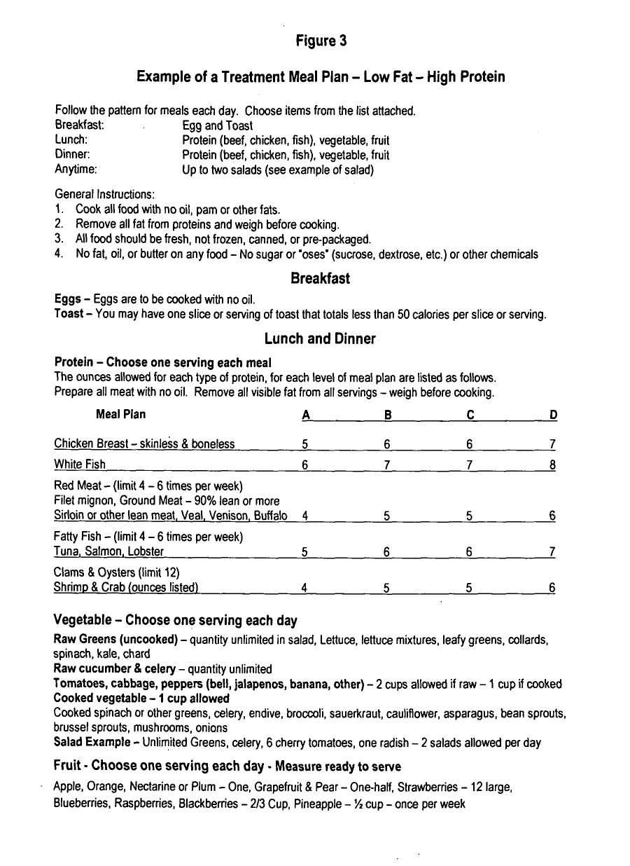

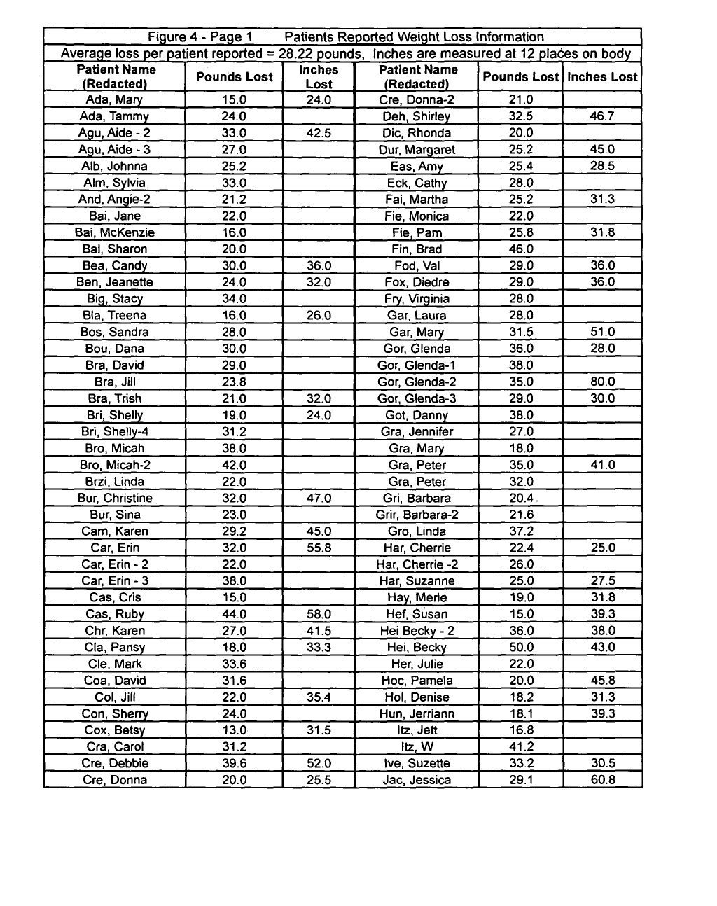

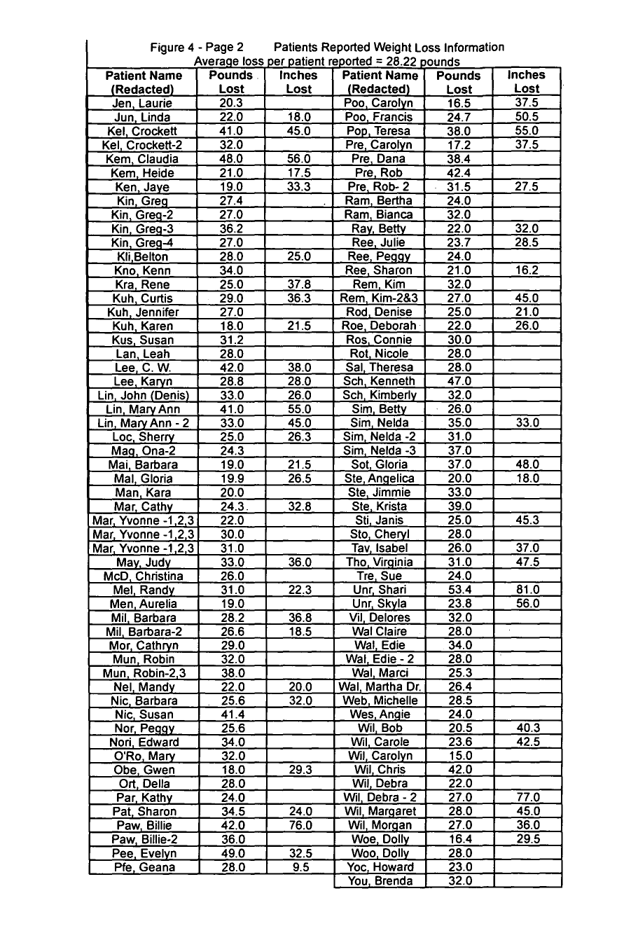

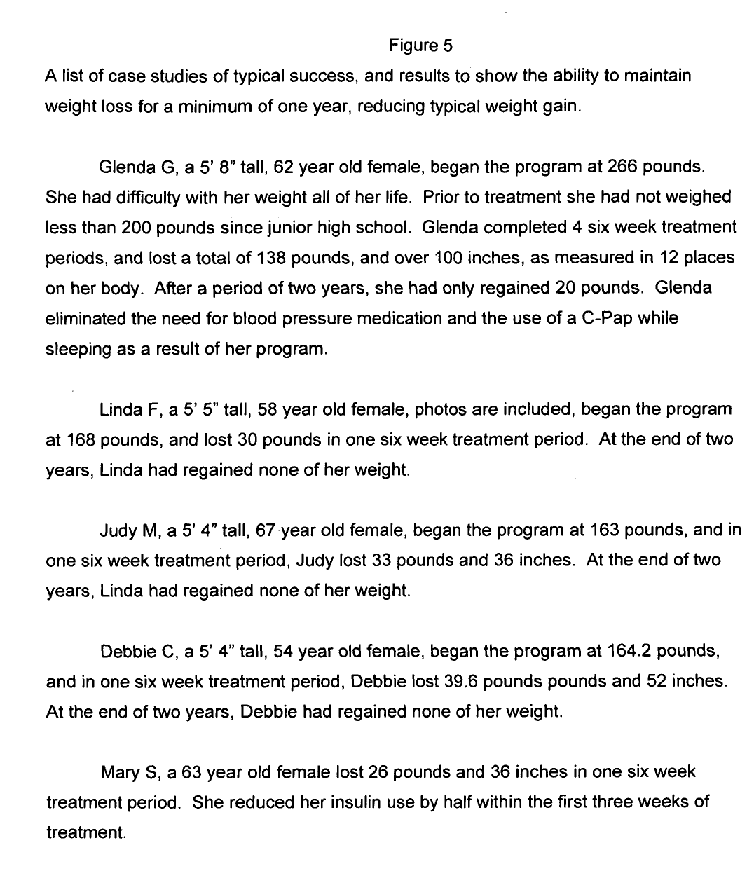

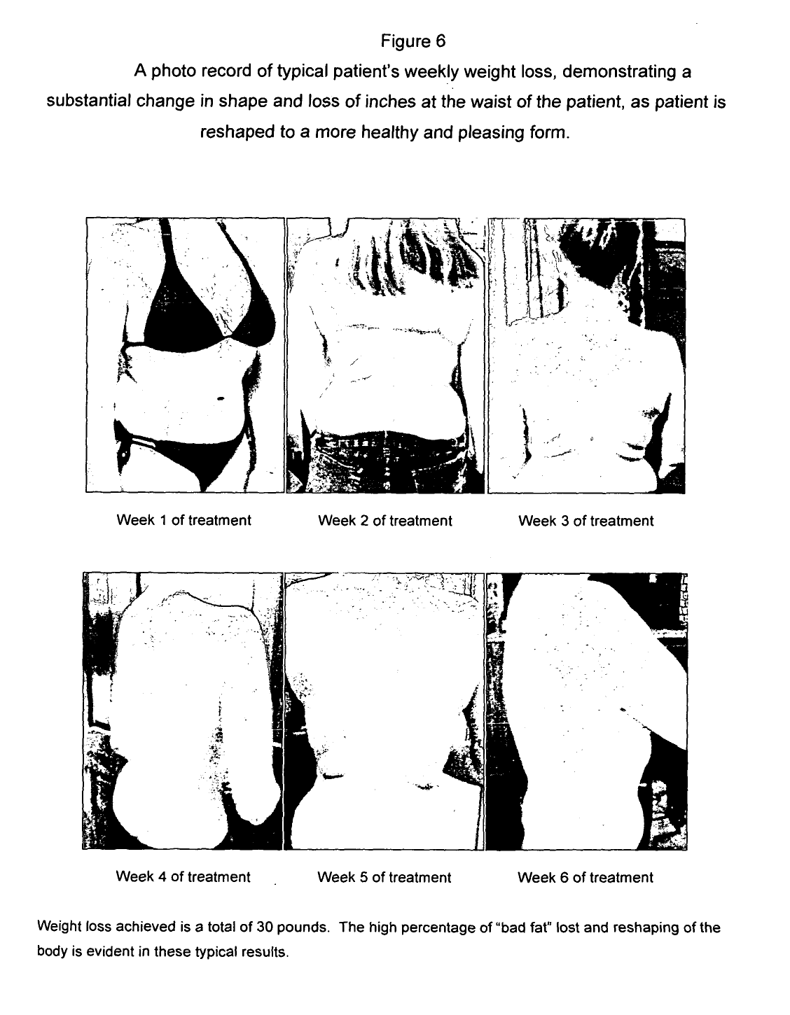

The “six week treatment period” may include a treatment meal plan, further described in FIG. 3, based upon the criteria described in FIG. 2. The physician will prescribe the meal plan appropriate to the patient, based on the criteria described. The physician will adjust the meal plan to a higher level of protein if patient is tired or hungry, and will reduce the plan to a lower level is patient is not hungry or tired, but has weight loss less than 0.75-1 pound per day if clinically obese. All meals to include only those items listed on FIG. 3 and to be prepared with no additional oils, sugar, or artificial sweeteners. Stevia may be used for a sweetener. Vinegar, lemon, lime, spices, tobacco, soy sauce, picante, and horseradish are allowed in moderation.

For the first two-three days of the six week treatment period only, the patient will add additional fats to their normal diet, in an amount to increase weight 1-3 pounds by day three of the treatment period.

No lotions, soaps or other products used on the skin, which contain oils, may be used during the “six week treatment period”, unless they include only mineral oil or petroleum products.

A prescription of HCG in a dosage of 250 IU is to be delivered subcutaneously or 375 IU of HCG delivered sublingually for six days a week for six weeks of the “six week treatment period”.

During the “six week treatment period”, the following is prescribed.
(a) One B6 Complex from Pure Encapsulations once daily each morning
(b) One Pure Encapsulation Magnesium Citrate capsule once daily prior to bedtime.
(c) Soluble fiber (compares to Benefiber) in an amount to exceed 50 grams daily, ingested within a 6 hour period, adjusted to patient tolerance
(d) Bowel toner (Dr. Schulze's Intestinal Formula #1) taken to relieve constipation when necessary, as directed by manufacturer to increase and encourage peristaltic action. Add one each day when constipated, stay at the dosage providing relief, and reduce daily dosage if consistency of bowel movements becomes liquid. Add one more Magnesium Citrate at bedtime for each Intestinal Formula #1 taken during the day.
(e) Large dose of B12 (5 mg) and Folic Acid (2.5 mg) to be delivered via subcutaneous or sublingual method, once weekly for the “six week treatment period”.
(f) No supplements containing oil are to be used during the “six week treatment period”.

Following the “six week treatment period” is a “three week stabilization period” in which no new carbohydrates are added to the treatment diet. Fats, additional protein, gravies, and other oils are to be added to meals to tolerance to assure the ending weight at the end of the “six week treatment period” is maintain. Weight is not to be allowed to go up or down. If weight goes up patient is to eat less, if down, patient is to eat more; immediately returning to the final treatment weight.

Essential fatty acids provided in Udo's oil, Omega 3-6-9 in a ration of 2:1:1 is recommended at this time.

Following the “three week stabilization period” a “three week return to normal foods” is accomplished by adding normal foods desired by patient carefully, being sure to maintain ending treatment weight.

Should patient feel tired or have headaches, assure good daily elimination of 1 cup or more of stool. If elimination is adequate, add protein to the treatment diet by moving to the next higher meal plan.

A patient with low weight loss, not fatigue, and no other negative symptoms may be instructed to use a meal plan one step lower in protein.

If all other issues are stable, a “protein only day” with protein of three times the portion allowed for one meal and no carbohydrates may encourage additional weight loss. Do not repeat this “protein only day” more than once per week.

Exercise is not required as a part of the program, however, if a patient chooses to exercise and becomes tired, increase meal plan to one step higher level of protein.

Patient is to drink 75 to 100 ounces of water daily during treatment and stabilization.

Measure weekly and weigh daily to assure continued loss of inches and weight.

If patient becomes hungry, and achieves a goal weight, increase protein during the treatment period to maintain new goal weight, and avoid further weight loss until the end of the treatment period.

### APPENDICES

The following and/or accompanying disclosure information is provided as non-limiting examples of features, functions, structures, associations, connections, methods, steps, benefits, consequences, and the like that may be included independently, in any open combination, and in any limited combinational form (consisting of) despite any language to the contrary, such as but not limited to “must” “always” “never” “certainly” and the like. Any dimensions provided are exemplary and functionally equivalent ranges that one skilled in the art would recognize after reading this disclosure are implied. Disclosure provided may be prophetic, even is asserted as otherwise.

Having thus described the invention, what is desired to be protected by Letters Patent, is presented in the subsequently appended claims:

1. A system and method for promoting, increasing, or facilitating a human patient that is desirous of losing weight or in need of a therapeutically effective method, combining and comprising a plurality of components to support the body during the stress of weight loss, wherein a meal plan provides adequate nutrition to avoid the typical plateau during weight loss, the use of fiber to remove toxins and waste, a simulant of peristaltic action in the bowel, a limited treatment time for weight loss, and a stabilizing period to establish a new weight and the return to normal meals.

2. A means according to claim 1 comprising administering an additional active ingredient for the treatment of weight loss to include human chorionic gonadotropin, via subcutaneous or intramuscular injection or sublingual dissolution, or any other method.

3. A means according to claim 1 comprising administering an additional active ingredient for the treatment of weight loss and support to include vitamins such as B12, and B12 and Folic.

4. A means according to claim 1 comprising administering an additional active ingredient to stimulate peristaltic activity in the bowel.

5. A means according to claim 1 comprising administering an additional active ingredient to support liver functions.

6. A means according to claim 1 comprising administering an additional active ingredient to support liver functions.

7. A means according to claim 1 comprising administering 75-100 ounces of water during weight loss treatment and stabilization.

8. A means according to claim 1 to reduce cellulite and reshape the body without weight loss.

9. A means according to claim 1 to add additional supplements to support overall metabolic function.

10. A means according to claim 1 to increase weight loss and comfort of patient by eating a high fat diet for the first 3 days of the treatment period.

11. A means according to claim 1 to increase protein to reduce hunger and fatigue.

12. A means according to claim 1 of reducing insulin usage in an insulin-dependent human patient.

13. A means according to claim 1 of reducing blood pressure and the use of blood pressure medication in a human patient with high blood pressure.

14. A means for providing reshaping of the body and increasing inches lost during weight loss.

15. A method of inhibiting rebound weight gain after weight loss by combining and comprising a plurality of components to support the body during the stress of weight loss, wherein a meal plan provides adequate nutrition to avoid the typical plateau during weight loss, the use of fiber to remove toxins and waste, a simulant of peristaltic action in the bowel, a limited treatment time for weight loss, and providing a stabilizing period to establish a new weight and the return to normal meals.

16. A method of reducing or eliminating hunger and fatigue during weight loss by combining and comprising a plurality of components to support the body during the stress of weight loss, wherein a meal plan provides adequate nutrition to avoid the typical plateau during weight loss, the use of fiber to remove toxins and waste, a simulant of peristaltic action in the bowel, a limited treatment time for weight loss, and providing a stabilizing period to establish a new weight and the return to normal meals.
